(19) United States
(12) Patent Application Publication
(10) Pub. No.: US 2006/0292223 A1
(43) Pub. Date: Dec. 28, 2006

(54) Gel compositions for topical administration

(75) Inventors: David Woolfson, Belfast (IE); Jim Mc Ilroy, Belfast (IE)

Correspondence Address: FITZPATRICK CELLA HARPER & SCINTO, 30 ROCKEFELLER PLAZA, NEW YORK, NY, 10112, US

(73) Assignee: WARNER CHILCOTT COMPANY INC,, Fajardo, PR (US)

(21) Appl. No.: 11/454,717

(22) Filed: Jun. 16, 2006

Related U.S. Application Data
(60) Provisional application No. 60/691,441, filed on Jun. 16, 2005.

(51) Int. Cl. A61K 9/14; A61K 31/56

(52) U.S. Cl. 424/486; 514/182

(57) ABSTRACT

Pharmaceutical gel compositions containing pharmacologically active agent for topical administration, as well as a method of making the same, are disclosed.

This application claims the benefit of U.S. Provisional Patent Application No. 60/691,441, filed Jun. 16, 2005, the entire disclosure of which is incorporated by reference herein.

### BACKGROUND OF THE INVENTION

1. Field of the Invention

This invention is directed to gel compositions for topical administration, as well as to methods of making and administering the same. The gel compositions contain a gelation promoter which at least partially solubilizes an active ingredient and which gels a polymeric component therein.

2. Related Background Art

Conventional semi-solid topical preparations comprise single-phase systems or two-phase emulsified systems. As used herein, the term “semi-solid” is understood to refer to the rheological properties of the compositions themselves, such that the compositions will flow under an applied force but will remain in situ following application to any accessible body surface.

Single-phase semi-solid systems may be hydrophobic ointments or hydrophilic gels. Two-phase semi-solid systems are emulsions, in which the continuous phase may be hydrophobic, as in a water-in-oil emulsion, or hydrophilic, as in an oil-in-water emulsion. For a pharmacologically active component of such preparations, it is preferred that such component is substantially in solution rather than in suspension within the preparation, due to the greater release rate of the pharmacologically active component from solution. For a pharmacologically active component whose aqueous solubility is relatively low, for example, where less than 25% of the total loading may be solubilized within an aqueous preparation, a hydrophobic preparation or a preparation with a hydrophobic phase is desirable. Thus, pharmacologically active components of low aqueous solubility may be solubilized within a hydrophobic ointment or a two-phase emulsion system in which it is primarily in solution within the oil phase.

A problem encountered with conventional ointments and water-in-oil hydrophobic preparations is that they can be greasy and/or extremely difficult to apply due to the significant hydrophobic oil or wax component. If applied onto the skin, such preparations tend to stain clothing and are preferably used only where the skin condition is extremely dry.

Oil-in-water preparations, sometimes referred to in the art as “vanishing” creams, overcome the problems of greasiness and staining of clothing. However, pharmacologically active components of lower aqueous solubility are solubilized within the internal oil emulsion phase of such preparations. They must, therefore, partition across the external aqueous (continuous) phase to reach their site of action. This may restrict release and subsequent topical bioavailability of the pharmacologically active component from such preparations.

Conventional gel preparations overcome many of the foregoing problems. As used herein, the term “gel” is understood to be a semi-solid matrix of particles interpenetrated by a liquid, in which the structural coherent matrix contains a high portion of liquid, usually water. Such gels comprise a single phase. Where the liquid phase is water, or substantially water, pharmacologically active components of low aqueous solubility will be present substantially in suspension; thus release and subsequent bioavailability may be restricted.

Known pharmaceutical gel compositions for topical administration conventionally comprise polymers, for example, modified cellulose ethers, natural gums or polymers containing pendant carboxylic acid groups, or their esters, or having pendant anhydrides of dicarboxylic acid groups, or mixtures thereof. Carboxylic acid polymers in aqueous systems are conventionally neutralized from a starting pH of about 2.5 to 3.5 to a pH of 4.0 or more in order to achieve gelation. Conventional neutralizers comprise sodium hydroxide, potassium hydroxide, ammonium hydroxide, arginine, aminomethyl propanol, tetrahydroxypropyl ethylenediamine, triethanolamine, tromethamine, PEG-15 cocamine, diisopropanolamine or triisopropanolamine. However, all polymer gel compositions, exemplified by the foregoing, will require the presence of a solubilizing agent to substantially solubilize a pharmacologically active agent of low aqueous solubility. For this reason, known pharmaceutical gel compositions for topical administration may conventionally contain an alcoholic component, ethanol, as a solubilizer. This may also present difficulties in certain clinical conditions such as dry skin.

More specifically, known pharmaceutical gel compositions for external application to the skin comprise Estrogel® (Solvay, US) and Sandrena® (Organon, Netherlands). Estrogel® is a hydro-ethanolic gel containing 0.06% estradiol; the excipients are ethanol, Carbomer 934 and triethanolamine, the balance being purified water. Sandrena® is another hydro-alcoholic gel containing 0.1% estradiol; its excipients are Carbomer 934, sodium hydroxide, propylene glycol, ethanol and water. Clearly, the base of these pharmaceutical gel compositions is a mixture of water and ethanol. The ethanol is intended to increase estrogen solubility in the gel and assist absorption into the stratum corneum.

EP-B-435200 in the names of Nitto Denko Corp. and Teikoku Hormone Mfg. Co. relates to an estrogen-containing gel for external administration to the skin. The gels are covalently cross-linked using, for example, a titanium or aluminum chelate compound. There is no disclosure or suggestion that a gelation promoter might be selected to both solubilize the estrogen and to gel the polymer.

EP-B-813412 in the name of Laboratoire Innothera relates to vaginal estradiol-containing gels comprising a polymer that has been gelled by neutralization by a conventional neutralizer. There is no disclosure or suggestion that a gelation promoter might be selected to both solubilize the estrogen and to gel the polymer in a non-aqueous or substantially non-aqueous environment.

DE-A-199 45 522 in the name of Hexal AG relates to compositions for topical administration. The compositions are oil-in-water emulsions containing a polymer as a thickening agent. The compositions are erroneously described in DE-A-199 45 522 as gels. There is no disclosure or suggestion that a gelation promoter might be selected to both solubilize the estrogen and to gel the polymer.

Accordingly, pharmaceutical gel compositions for topical administration which do not suffer from the deficiencies of conventional semi-solid topical compositions are highly desirable.

### SUMMARY OF THE INVENTION

The present invention is directed to a pharmaceutical gel composition for topical administration comprising (a) at least one pharmacologically active agent in an amount of about 0.00001% to about 10% by weight of the composition; (b) at least one hydrogen-bonding gelation polymer; and (c) at least one gelation promoter in an amount effective to at least partially solubilize the pharmacologically active agent and to gel the polymer, wherein at least a portion of the pharmacologically active agent is dissolved in the composition at 15° C. The formation of a gel from one or more suitable hydrogen-bonding gelation polymer(s) capable of viscosity enhancement in the presence of at least one gelation promoter capable of both causing gelation promotion and at least partial solubilization of the at least one pharmacologically active agent is desirable for both improved release rate of the drug from the product and for a more elegant water-washable product. In preferred embodiments, the hydrogen-bonding gelation polymer is present in an amount sufficient to form a gel with a viscosity ranging from about 25 Pa·s to about 1000 Pa·s at 20° C. In other preferred embodiments, at least 25%, more preferably at least 50%, and most preferably at least 75% of the pharmacologically active agent is dissolved at 15° C. In certain embodiments of the present invention, the at least one pharmacologically active agent is selected from agents that are cosmetically, therapeutically or prophylactically active in the following cosmetic, therapeutic and prophylactic areas: gynecology, urinary tract disorders, infection control, inflammatory conditions, central nervous system disorders and skin disorders. In certain embodiments, the at least one hydrogen-bonding gelation polymer is a homopolymer, copolymer or interpolymer having pendant carboxylic acid groups, having pendant anhydrides of dicarboxylic acid groups, or having both (or esters of any thereof). In certain embodiments, the at least one gelation promoter is at least one polyhydric alcohol, at least one polyglycol or a combination thereof. In certain embodiments, the gelation promoter comprises an aqueous solution of a gelation promoter.

The present invention is further directed to a method of making pharmaceutical gel compositions for topical administration comprising the step of admixing at least one pharmacologically active agent in an amount of about 0.00001% to about 10% by weight of the composition, at least one hydrogen-bonding gelation polymer, and at least one gelation promoter or aqueous solution thereof in an amount effective to at least partially solubilize the pharmacologically active agent and to gel the polymer to form the pharmaceutical gel composition, wherein at least a portion of the pharmacologically active agent is dissolved in the composition at 15° C. In a preferred embodiment, the admixing comprises (a) at least partially solubilizing the pharmacologically active agent in the gelation promoter (or in the aqueous solution of the gelation promoter) to form an at least partially solubilized pharmacologically active agent preparation; and (b) combining the at least partially solubilized pharmacologically active agent preparation with the hydrogen-bonding gelation polymer to form the pharmaceutical gel composition.

The present invention is still further directed to a pharmaceutical gel composition made according to the present inventive method and to a method of administering a pharmaceutical gel composition of the present invention.

### DETAILED DESCRIPTION OF THE INVENTION

As used herein, the term “topical administration” refers to administration onto any accessible body surface of any human or animal species, preferably the human species, for example, the skin or mucosal epithelia such as nasal or rectal epithelia. In certain embodiments of this invention, “topical” refers to an external application to a skin surface.

As used herein, “pharmacologically active agent” or “agent” or “drug” or “active agent” or “active ingredient”, etc., refers to any agent capable of defending against, or treating, a disease or cosmetic state in the human or animal body, or a prodrug thereof. Such pharmacologically active agents may be organic or inorganic and may be prophylactically or therapeutically active. Alternatively or additionally, such pharmacologically active agents may be cosmetically active. As used herein, “prophylactically active” refers to an agent's (or its prodrug's) effectiveness in defending against a disease state in the human or animal body, preferably the human body. As used herein, “therapeutically active” refers to an agent's (or its prodrug's) effectiveness in treating a disease state in the human or animal body, preferably the human body. As used herein, “cosmetically active” refers to an agent's (or its prodrug's) effectiveness in defending against or treating a cosmetic condition in or on the human or animal body, preferably the human body.

Without being bound by theory, as used herein, “hydrogen-bonding gelation polymer” refers to polymers, which are capable of taking part in hydrogen bonding with the functional groups of a gelation promoter. Such polymers may also be capable of gelation by neutralization but, in the compositions of the present invention, gelation is achieved by hydrogen-bonding. As used herein, “hydrogen-bonding” refers to a non-covalent bond between hydrogen and another atom, usually nitrogen or oxygen. Hydrogen bonding does not involve the sharing of electrons between the bonded atoms and, therefore, does not satisfy the valence of either atom.

As used herein, “gelation promoter” refers to a substance, or an aqueous solution thereof, having at least two functional groups which, it is thought, can take part in hydrogen bonding with a gelation polymer to accomplish uncoiling and/or cross-linking of a polymer chain and also acts as a solubilizer of the at least one pharmacologically active agent. Such functional groups comprise hydroxy or ethoxy (defined as —O— or ether links) groups, or a mixture thereof. The term “gelation promoter” excludes any substance having a single functional group, which could possibly take part in hydrogen-bonding. Such excluded substances include ethanol.

The first embodiment of the present invention is a pharmaceutical gel formulation for topical administration comprising at least one, at least partially solubilized, pharmacologically active agent, at least one hydrogen-bonding gelation polymer, and at least one gelation promoter. As used herein, “at least partially solubilized” refers to at least a portion of the pharmacologically active agent being dissolved; more specifically, it refers to at least 25%, more preferably at least 50%, and most preferably at least 75%, of the pharmacologically active agent being dissolved at 15° C. In a preferred embodiment of this invention, the pharmacologically active agent is “substantially solubilized”; more specifically, it refers to at least 50%, more preferably at least 60%, and most preferably at least 90%, of the pharmacologically active agent being dissolved at 15° C. The units % relate to % w/w, so that “at least 25%” requires that at least a quarter, by weight, of the added pharmacologically active agent is dissolved in the composition at 15° C. For purposes of any of the first to fourth embodiments of this invention, it is to be understood that “dissolved” can refer to either or both of dissolution in the composition as a whole or dissolution in the gelation promoter or an aqueous solution of the gelation promoter.

An exemplary method of measuring the proportion of dissolved pharmacologically active agent is based on the solubility of the pharmacologically active agent in the gelation promoter (or an aqueous solution thereof) at 15° C. The solubility in the gelation polymer is calculated by preparing saturated solutions of the pharmacologically active agent in the gelation promoter at 15° C. (in triplicate). These saturated solutions were prepared by adding the pharmacologically active agent to the gelation promoter at 15° C. until saturation was achieved (i.e., no more pharmacologically active agent dissolved in the gelation promoter). The saturated solutions were then placed in a shaker at 15° C. for 12 hours, after which time more pharmacologically active agent was added if required. Finally, the saturated solutions were centrifuged at 15° C. and the supernatant analyzed by HPLC to determine the amount of dissolved pharmacologically active agent in the gelation promoter or an aqueous solution thereof. The solubility of the pharmacologically active agent in the composition at 15° C. is measured by first centrifuging the composition under centrifugation conditions sufficient to remove any suspended pharmacologically active agent, and then extracting the pharmacologically active agent from the supernatant using a suitable solvent or solvent mixture. The solvent extract is then analyzed by HPLC to determine the amount of dissolved pharmacologically active agent in the composition at 15° C.

The at least one pharmacologically active agent is employed in an amount ranging from about 0.00001% to about 10%, preferably in an amount ranging from about 0.0025% to about 6%, and more preferably in an amount ranging from about 0.0045% to about 5% by weight of the composition.

Suitable pharmacologically active agents include, but are not limited to, those which are classified as being cosmetically, therapeutically or prophylactically active in the following cosmetic, therapeutic and prophylactic areas: gynecology, urinary tract disorders, infection control, inflammatory conditions, central nervous system disorders and skin disorders.

More specifically, suitable pharmacologically active agents (prophylactic, therapeutic and/or cosmetic) include, without limitation:

those drugs active in the field of gynecology such as desogestrel, etonogestrel, medroxyprogesterone, medroxyprogesterone acetate, mestranol, nonoxynol-9, tibolone, salts or esters thereof; contraceptive and/or hormone replacement therapy drugs such as dehydroepiandrosterone sulfate, dienestrol, diethylstilberol, estrogens such as estradiol, estriol, estradiol-3-acetate and ethinyl estradiol, gestodene, levonorgestrel, luteinizing hormone releasing hormone, norethisterone, norethisterone acetate, norgestimate, progesterone, ST-1435, testosterone, testosterone acetate, salts or esters thereof; drugs for cervical ripening/induction of labor such as misoprostol, oxytocin, PGE2, dinoprostone, salts or esters thereof; drugs for treatment of endometriosis such as danazol, salts or esters thereof; osteoporosis and/or hormone replacement therapy drugs such as selective estrogen receptor modulators (SERMs), for example, raloxifene, salts and esters thereof; those drugs active in the field of central nervous system disorders including pain and migraine drugs such as 5HT-1 receptor blockers, for example, sumatriptan, salts or esters thereof; drugs for anxiety, depression and premenstrual syndrome such as selective serotonin reuptake inhibitors (SSR1s), for example, fluoxetine, antiemetic drugs such as ondansetron, salts and esters thereof;

those drugs active in the field of urinary tract disorders such as tolterodine tartrate, oxybutynin, salts and esters thereof;

those drugs active in the field of infection control, for example, antibacterial drugs such as clindamycin, doxycycline, erythromycin, salts or esters thereof; antifungal drugs such as clotrimazole, fluconazole, terconazole, salts or esters thereof; antimalarial agents; antiprotozoal agents; antiviral drugs, including antiretroviral agents such as acyclovir, famciclovir, valaciclovir, saquinavir, nevirapine, salts or esters thereof;

those drugs active in the field of skin disorders such as hydrocortisone, beclomethasone, betamethasone, clobetasol, fluocinolone, triamcinolone, vitamins and vitamin analogs, benzyl peroxide, salts or esters thereof; those drugs active in the field of inflammatory conditions, including non-steroidal anti-inflammatory drugs such as ibuprofen, diclofenac, ketoprofen, flurbiprofen, salts or esters thereof.

The use of pharmacologically active agents other than those specifically listed above, i.e., either other non-listed pharmacologically active agents which fall within the above-enumerated classes or non-listed pharmacologically active agents which fall outside the above-enumerated classes, are contemplated and thought suitable for use in the present invention. In other words, the present invention is not limited to the specifically listed pharmacologically active agents.

Suitable drugs also include their prodrugs, salts and esters where appropriate. Some of the drugs mentioned hereinabove may be suitable for use in more than one cosmetic, therapeutic or prophylactic area; for example, those drugs active in infection control may also be useful in skin disorders.

In certain embodiments of the present invention, the at least one pharmacologically active agent is an estrogen, more preferably an estrogen selected from the group comprising 17β-estradiol, mestranol, conjugated estrogens USP, estrone, or ethinyl estradiol or salts, esters or prodrugs thereof. Other suitable estrogens include those described in each of U.S. patent application Ser. Nos. 11/009,617 and 11/009,618 [Attorney Docket Nos. 02911.004500 and 02911.004400, respectively], each filed on Dec. 10, 2004, and those described in each of U.S. Provisional Patent Application Nos. 60/698,865 and 60/698,866 [Attorney Docket Nos. 02911.006500 and 02911.006900, respectively], each filed on Jul. 12, 2005. The disclosures of each of these applications are incorporated in their entirety by reference herein. The estrogen, if present, is included in the pharmaceutical composition of the present invention in an amount ranging from about 0.00001% to about 2%, more preferably from about 0.0005% to about 0.05%, still more preferably from about 0.00075% to about 0.025%, by weight of the composition. When the at least one pharmacologically active agent is an estrogen, the pharmaceutical gel compositions of the present invention may contain other pharmacologically active agents. Other such suitable active ingredients include, without limitation, other steroids such as a progestogen (for example, progestogen and its derivatives such as 17-hydroxy progestogen esters and 19-nor-17-hydroxy progestogen esters, norgestrel, norgestimate, demegestone, drospirenone, dydrogesterone, medrogestone, medroxy progesterone and esters thereof such as medroxy progesterone acetate, norethesterone, norethindrone, norethindrone acetate, levonorgestrel, desogestrel, 3-ketodesogestrel, gestodene and the like) or an androgen (such as testosterone, esters thereof, methyl-testosterone and prodrugs and combinations thereof), in an amount appropriate for clinical efficacy.

Hydrogen-bonding gelation polymers suitable for use in the present invention include, without limitation, homopolymers, copolymers and interpolymers having pendant carboxylic acid groups and/or having pendant anhydrides of dicarboxylic acid groups, or esters of any thereof, such as polyacrylic acid derivatives or copolymers of acrylic acid with long-chain alkyl acrylates (e.g., those sold under the tradename Carbopol® (Noveon, US)) or polymethyl vinyl ether/maleic anhydride copolymers (e.g., those commercially available under the tradename Gantrez® (ISP, US)) and combinations thereof. The hydrogen-bonding gelation polymer may be cross-linked or not. While acrylic acid is the most common primary monomer, other suitable monomers include all α-β unsaturated monomers with carboxylic pendant groups or anhydrides of dicarboxylic acids (see, U.S. Pat. No. 5,349,030, the contents of which are incorporated herein by reference).

The pharmaceutical gel compositions of the present invention may contain, as the hydrogen-bonding gelation polymer, at least one poly(acrylic) acid (carbomer) or a mixture thereof. Lightly crosslinked poly(acrylic) acid polymers, commercially available in a range of viscosities as Carbopol® are suitable. Poly(acrylic) acid polymers with a greater degree of crosslinking, commercially available as Noveon® are also suitable. Mixtures of the aforementioned polymers are also suitable. Carbopol® polymers are preferred. Whilst it will be apparent that many such polymers may be employed in the present invention, Carbopol® polymers with a Brookfield viscosity (measured at 25° C. at 20 rpm using 0.5% (w/w) aqueous solution) of between 3,000 and 15,000 cP are preferred—suitable examples are Carbopol® 941NF, Carbopol® 981NF, Carbopol® 971NF and Carbopol® ETD2050. Carbopol® 974P is most preferred.

The hydrogen-bonding gelation polymer is included in the pharmaceutical gel composition in an amount to give a viscosity of between about 25 Pa·s and about 1000 Pa·s at 20° C., more preferably between about 40 Pa·s and about 500 Pa·s at 20° C.

The gelation promoter of the present invention, in theory, aids in gel formation by uncoiling polymer chains by supplying functional groups (hydroxyl groups or ether links) which are capable of participating in hydrogen bonding with the carboxylic acid groups on the backbone of the hydrogen-bonding gelation polymer. Without being bound by theory, gelation is subsequently thought to occur via linear chain entanglement or cross-linking, depending on the nature of the gelation polymers. According to the present invention, the gelation promoter must have at least two functional groups capable of participating in hydrogen bonding with the hydrogen-bonding gelation polymer. Suitable gelation promoters therefore include, without limitation, polyhydric alcohols, polyglycols, and combinations thereof. Preferred gelation promoters include glycerol, propylene glycol and low molecular weight polyethylene glycols which remain as liquids as room temperature, i.e., polyethylene glycol 200 to 700, for example, polyethylene glycol 400.

Exemplary polyhydric alcohols include, without limitation, 1,2-butanediol, 1,3-butanediol, 1,4-butanediol, butynediol, butenediol, diethylene glycol, ethylene glycol, glycerol, glycofurol, 1,2-hexanediol, 1,2,6-hexanetriol, 3-methyl-1,5-pentanediol, 2-methyl-1,3-propanediol, 1,9-nonanediol, 1,5-pentanediol, poly(vinyl alcohol), 1,3-propanediol and propylene glycol. In addition, solutions of solid polyhydric alcohols could be used.

Exemplary polyglycols include, without limitation, butyl glycol, butyl diglycol, butyl polyglycol, diethylene glycol monomethyl ether, diethylene glycol dimethyl ether, diethylene glycol monoethyl ether, diethylene glycol diethyl ether, dipropylene glycol, dipropylene glycol dimethyl ether, poloxamers, methyl diglycol, methyl triglycol, methyl tetraglycol, poly(ethylene glycol), poly(oxyethylene) alkyl ethers, poly(oxyethylene) alkyl esters, poly(propylene glycol), tetraethylene glycol dimethyl ether, triethylene glycol, triethyl glycol dimethyl ether, tripropylene glycol, and glycol-silane copolymers. In certain preferred embodiments, the gelation promoter is polyethylene glycol. In other preferred embodiments, the gelation promoter is a poloxamer, i.e., a copolymer of polyoxyethylene and polyoxypropylene. In other preferred embodiments, the gelation promoter is selected from the group comprising propylene glycol, polyethylene glycol (such as PEG 400), glycerol and diethylene glycol monoethyl ether.

The at least one gelation promoter is present in the pharmaceutical gel composition of the present invention in an amount effective to gel the hydrogen-bonding gelation polymer and to at least partially (and preferably substantially) solubilize the pharmacologically active agent. The gelation promoter may comprise one of the materials noted above or an aqueous solution of one of the materials noted above, or a mixture thereof. When the gelation promoter is incorporated as an aqueous solution, the aqueous solvent may be water (most preferred) or some combination of water and water-miscible solvents.

In view of the importance of avoiding conventional drug solubilizing agents in the pharmaceutical gel compositions of the present invention, the pharmaceutical compositions are substantially free of drug solubilizing agents such as ethanol. As used herein, the term “substantially free” is understood to be less than about 0.05% of said drug solubilizing agent, preferably less than about 0.005% of said drug solubilizing agent, still more preferably less than about 0.001% of said drug solubilizing agent, by weight of the composition. In a particular embodiment, the term “substantially free” may be understood to be less than about 0.05% of ethanol, preferably less than about 0.005% of ethanol, still more preferably less than about 0.001% of ethanol, by weight of the composition. All % units are w/w.

Without wishing to be bound by theory, it is believed that the hydrogen-bonding gelation polymer forms hydrogen bonds with the gelation promoter, resulting in thickening of the matrix without resorting to conventional neutralizers. The gel matrix so achieved is a clear, or almost clear, transparent matrix. This is desirable from the user's point of view. The gel matrix so achieved is, in itself, bioadhesive, more specifically mucoadhesive, in that the gel matrix possesses the property of being able to persist at an epithelial surface by, it is believed, polymer entanglement with surface mucin and/or non-covalent bond formation between the polymer(s) of the gel matrix and surface mucin. This is desirable for topical delivery.

The pharmaceutical composition of the present invention may also contain any pharmaceutically acceptable excipient, as desired. When present, such pharmaceutically acceptable excipients are included in an amount which can be readily determined by one of ordinary skill in the art. Suitable excipients include, without limitation, waxes (such as white soft paraffin), poly(vinyl alcohol), hydroxyethylcellulose, hydroxypropylcellulose, hydroxypropylmethylcellulose, suitable preservatives including, without limitation, para-hydroxy benzoate compounds, buffers (for example, those buffers comprising weak organic acids such as lactic acid or acetic acid) and combinations thereof.

The second embodiment comprises a method of making a pharmaceutical gel composition for topical administration comprising the step of admixing at least one pharmacologically active agent in an amount of about 0.00001% to about 10% by weight of the composition, at least one hydrogen-bonding gelation polymer, and at least one gelation promoter or aqueous solution thereof in an amount effective to at least partially solubilize the pharmacologically active agent and to gel the polymer to form the pharmaceutical gel composition, wherein at least a portion of the pharmacologically active agent is dissolved in the composition at 15° C. The ingredients can be admixed using any suitable means. Typically, any mixing step is accomplished in a suitable vessel with vigorous agitation, i.e., high shear mixing. According to a preferred embodiment of the inventive method, the admixing is accomplished by at least partially solubilizing the pharmacologically active agent in the gelation promoter (or aqueous solution thereof) to form an at least partially (and preferably a substantially) solubilized pharmacologically active agent preparation and then combining the at least substantially solubilized pharmacologically active agent preparation with the hydrogen-bonding gelation polymer to form the pharmaceutical gel composition.

Optional additional steps include those which result in the addition of one or more of another pharmacologically active agent(s) and pharmaceutically acceptable excipient(s). The details regarding the pharmacologically active agent, hydrogen-bonding gelation polymer and gelation promoter, i.e., type and amount, as well as the details regarding other possible ingredients, are as set forth above with regard to the first embodiment of this invention.

An additional embodiment of the present invention is directed to a pharmaceutical gel composition made according to the method of the second embodiment of the invention.

Still another embodiment of the present invention is directed to a method of topical administration of a pharmacologically active agent for a human or animal comprising the step of administering the pharmaceutical gel composition of the present invention to an accessible body surface of the human or animal, such as to the skin or mucosal epithelia such as nasal or rectal epithelia.

Specific embodiments of the invention will now be demonstrated by reference to the following examples. It should be understood that these examples are disclosed solely by way of illustrating the invention and should not be taken in any way to limit the scope of the present invention.

### EXAMPLE 1

### Anhydrous Gelation Promoter/Carbomer/Estradiol Formulation

A single-phase pharmaceutical gel composition was made to contain the components set forth in Table 1 below.

TABLE 1

| Ingredient | % w/w |
|---|---|
| Carbomer (Carbopol 974P) | 2.50 |
| Glycerol | 97.49 |
| 17β-estradiol | 0.01 |

The 17β-estradiol was solubilized in a stock solution of glycerol and then added to the remainder of the glycerol. Then, the carbomer was added and mixed with high shear until gelation occurred.

Gel viscosity was determined using a TA Advanced Rheometer AR550 in stepped flow mode, with a time constant of 10 seconds. Samples (3 replicates) were loaded between a set of 40 mm standard parallel plates, with a plate gap of 1000 microns. Each sample was allowed to equilibrate for 2 minutes before the shear stress was applied. A fresh sample was applied for each replicate analysis. The shear stress was increased from 50-250 Pa, and the viscosity was determined by application of the Power Law Model to the resulting flow rheogram. All analyses were performed at a controlled temperature of 20° C. A pharmaceutical gel composition having a viscosity of 199.2±17.1 Pa·s at 20° C. was obtained.

### EXAMPLE 2

### Anhydrous Gelation Promoter/Carbomer/Testosterone Formulation

A single-phase pharmaceutical gel composition was made to contain the components set forth in Table 2 below.

TABLE 2

| Ingredient | % w/w |
|---|---|
| Carbomer (Carbopol 974P) | 2.500 |
| Propylene Glycol | 97.485 |
| Testosterone | 0.015 |

The testosterone was solubilized in a stock solution of propylene glycol and then added to the remainder of the propylene glycol. Then, the carbomer was added and mixed with high shear until gelation occurred. A pharmaceutical gel composition having a viscosity of 187.8±7.7 Pa·s at 20° C. was obtained (as determined using the method of Example 1).

### EXAMPLE 3

### Anhydrous Gelation Promoter/Carbomer/Progesterone Formulation

A single-phase pharmaceutical gel composition was made to contain the components set forth in Table 3 below.

TABLE 3

| Ingredient | % w/w |
|---|---|
| Carbomer (Carbopol 974P) | 2.50 |
| Propylene Glycol | 97.48 |
| Progesterone | 0.02 |

The progesterone was solubilized in a stock solution of propylene glycol and then added to the remainder of the propylene glycol. Then, the carbomer was added and mixed with high shear until gelation occurred. A pharmaceutical gel composition having a viscosity of 215.2±24.3 Pa·s at 20° C. was obtained (as determined using the method of Example 1).

### EXAMPLE 4

### Anhydrous Gelation Promoter/Carbomer/Norethisterone Acetate/Estradiol Formulation

A single-phase pharmaceutical gel composition was made to contain the components set forth in Table 4 below.

TABLE 4

| Ingredient | % w/w |
|---|---|
| Carbomer (Carbopol 974P) | 2.500 |
| Propylene Glycol | 97.475 |
| 17β-estradiol | 0.005 |
| Norethisterone acetate | 0.020 |

17β-estradiol and norethisterone acetate were solubilized in a stock solution of propylene glycol and then added to the remainder of the propylene glycol. Then, the carbomer was added and mixed with high shear until gelation occurred. A pharmaceutical gel composition having a viscosity of 196.6±16.2 Pa·s at 20° C. was obtained (as determined using the method of Example 1).

### EXAMPLE 5

### Anhydrous Gelation Promoter/Carbomer/Oxybutynin Formulation

A single-phase pharmaceutical gel composition was made to contain the components set forth in Table 5 below.

TABLE 5

| Ingredient | % w/w |
|---|---|
| Carbomer (Carbopol 974P) | 2.5 |
| Propylene Glycol | 97.0 |
| Oxybutynin free base | 0.5 |

The oxybutynin was solubilized in a stock solution of propylene glycol and then added to the remainder of the propylene glycol. Then, the carbomer was added and mixed with high shear until gelation occurred. A pharmaceutical gel composition having a viscosity of 401.0±15.6 Pa·s at 20° C. was obtained (as determined using the method of Example 1).

### EXAMPLE 6

### Anhydrous Gelation Promoter/Carbomer/Doxycycline Formulation

A single-phase pharmaceutical gel composition was made to contain the components set forth in Table 6 below.

TABLE 6

| Ingredient | % w/w |
|---|---|
| Carbomer (Carbopol 974P) | 2.5 |
| Glycerol | 96.5 |
| Doxycycline free base | 1.0 |

The doxycycline was solubilized in a stock solution of glycerol and then added to the remainder of the glycerol. Then, the carbomer was added and mixed with high shear until gelation occurred. A pharmaceutical gel composition having a viscosity of 308.1±4.6 Pa·s at 20° C. was obtained (as determined using the method of Example 1).

### EXAMPLE 7

### Anhydrous Gelation Promoter/Carbomer/Doxycycline Formulation

A single-phase pharmaceutical gel composition was made to contain the components set forth in Table 7 below.

TABLE 7

| Ingredient | % w/w |
|---|---|
| Carbomer (Carbopol 974P) | 2.5 |
| Propylene Glycol | 97.4 |
| Doxycycline free base | 0.1 |

The doxycycline was solubilized in a stock solution of propylene glycol and then added to the remainder of the propylene glycol. Then, the carbomer was added and mixed with high shear until gelation occurred. A pharmaceutical gel composition having a viscosity of 120.2±13.9 Pa·s at 20° C. was obtained (as determined using the method of Example 1).

### EXAMPLE 8

### Anhydrous Gelation Promoter/Carbomer/Clindamycin HCl Formulation

A single-phase pharmaceutical gel composition was made to contain the components set forth in Table 8 below.

TABLE 8

| Ingredient | % w/w |
|---|---|
| Carbomer (Carbopol 974P) | 2.5 |
| Propylene Glycol | 96.5 |
| Clindamycin HCl | 1.0 |

The clindamycin was solubilized in a stock solution of propylene glycol and then added to the remainder of the propylene glycol. Then, the carbomer was added and mixed with high shear until gelation occurred. A pharmaceutical gel composition having a viscosity of 94.2±22.6 Pa·s at 20° C. was obtained (as determined using the method of Example 1).

### EXAMPLE 9

### Anhydrous Gelation Promoter/Carbomer/Erythromycin Formulation

A single-phase pharmaceutical gel composition was made to contain the components set forth in Table 9 below.

TABLE 9

| Ingredient | % w/w |
|---|---|
| Carbomer (Carbopol 974P) | 2.5 |
| Propylene Glycol | 96.5 |
| Erythromycin free base | 1.0 |

The erythromycin was solubilized in a stock solution of propylene glycol and then added to the remainder of the propylene glycol. Then, the carbomer was added and mixed with high shear until gelation occurred. A pharmaceutical gel composition having a viscosity of 452.7±63.8 Pa·s at 20° C. was obtained (as determined using the method of Example 1).

### EXAMPLE 10

### Anhydrous Gelation Promoter/Carbomer/Betamethasone Formulation

A single-phase pharmaceutical gel composition was made to contain the components set forth in Table 10 below.

TABLE 10

| Ingredient | % w/w |
|---|---|
| Carbomer (Carbopol 974P) | 2.5 |
| Propylene Glycol | 97.4 |
| Betamethasone 17,21 dipropionate | 0.1 |

The betamethasone 17,21 dipropionate was solubilized in a stock solution of propylene glycol and then added to the remainder of the propylene glycol. Then, the carbomer was added and mixed with high shear until gelation occurred. A pharmaceutical gel composition having a viscosity of 208.0±6.4 Pa·s at 20° C. was obtained (as determined using the method of Example 1).

### EXAMPLE 11

### Anhydrous Gelation Promoter/Carbomer/Terconazole Formulation

A single-phase pharmaceutical gel composition was made to contain the components set forth in Table 11 below.

TABLE 11

| Ingredient | % w/w |
|---|---|
| Carbomer (Carbopol 974P) | 2.5 |
| Glycerol | 96.5 |
| Terconazole free base | 1.0 |

The terconazole was solubilized in a stock solution of glycerol and then added to the remainder of the glycerol. Then, the carbomer was added and mixed with high shear until gelation occurred. A pharmaceutical gel composition having a viscosity of 436.4±3.9 Pa·s at 20° C. was obtained (as determined using the method of Example 1).

### EXAMPLE 12

### Anhydrous Gelation Promoter/Carbomer/Acyclovir Formulation

A single-phase pharmaceutical gel composition was made to contain the components set forth in Table 12 below.

TABLE 12

| Ingredient | % w/w |
|---|---|
| Carbomer (Carbopol 974P) | 2.5 |
| Diethylene glycol monoethyl ether | 92.5 |
| Acyclovir free base | 5.0 |

The acyclovir was solubilized in a stock solution of diethylene glycol monoethyl ether and then added to the remainder of the diethylene glycol monoethyl ether. Then, the carbomer was added and mixed with high shear until gelation occurred. A pharmaceutical gel composition having a viscosity of 176.1±41.0 Pa·s at 20° C. was obtained (as determined using the method of Example 1).

### EXAMPLE 13

### Anhydrous Gelation Promoter/Carbomer/Doxycycline/Benzyl Peroxide Formulation

A single-phase pharmaceutical gel composition was made to contain the components set forth in Table 13 below.

TABLE 13

| Ingredient | % w/w |
|---|---|
| Carbomer (Carbopol 974P) | 2.5 |
| Propylene glycol | 91.5 |
| Doxycycline free base | 1.0 |
| Benzyl peroxide | 5.0 |

Doxycycline and benzyl peroxide were solubilized in a stock solution of propylene glycol and then added to the remainder of the propylene glycol. Then, the carbomer was added and mixed with high shear until gelation occurred. A pharmaceutical gel composition having a viscosity of 162.3±34.9 Pa·s at 20° C. was obtained (as determined using the method of Example 1).

### EXAMPLE 14

### Anhydrous Gelation Promoter/Carbomer/Ondansetron Formulation

A single-phase pharmaceutical gel composition was made to contain the components set forth in Table 14 below.

TABLE 14

| Ingredient | % w/w |
|---|---|
| Carbomer (Carbopol 974P) | 2.5 |
| Propylene Glycol | 95.5 |
| Ondansetron free base | 2.0 |

The ondansetron was solubilized in a stock solution of propylene glycol and then added to the remainder of the propylene glycol. Then, the carbomer was added and mixed with high shear until gelation occurred. A pharmaceutical gel composition having a viscosity of 43.9±16.6 Pa·s at 20° C. was obtained (as determined using the method of Example 1).

### EXAMPLE 15

### Anhydrous Gelation Promoter/Carbomer/Ibuprofen Formulation

A single-phase pharmaceutical gel composition was made to contain the components set forth in Table 15 below.

TABLE 15

| Ingredient | % w/w |
|---|---|
| Carbomer (Carbopol 974P) | 2.5 |
| Propylene Glycol | 94.5 |
| Ibuprofen | 3.0 |

The ibuprofen was solubilized in a stock solution of propylene glycol and then added to the remainder of the propylene glycol. Then, the carbomer was added and mixed with high shear until gelation occurred. A pharmaceutical gel composition having a viscosity within the desired specification was obtained.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A pharmaceutical gel composition for topical administration comprising:
(a) at least one pharmacologically active agent in an amount of about 0.00001% to about 10% by weight of the composition;
(b) at least one hydrogen-bonding gelation polymer; and
(c) at least one gelation promoter in an amount effective to gel the polymer and to at least partially solubilize the pharmacologically active agent, wherein at least a portion of the pharmacologically active agent is dissolved in the composition at 15° C.

2. The pharmaceutical gel composition of claim 1, wherein topical administration is external administration to the skin.

3. The pharmaceutical gel composition of claim 1, wherein the at least one pharmacologically active agent is selected from agents that are active in a cosmetic, therapeutic or prophylactic area selected from the group consisting of gynecology, urinary tract disorders, infection control, inflammatory conditions, central nervous system disorders and skin disorders.

4. The pharmaceutical gel composition of claim 3, wherein the at least one pharmacologically active agent is an estrogen.

5. The pharmaceutical gel composition of claim 4, wherein the estrogen is selected from the group consisting of 17β-estradiol, mestranol, conjugated estrogens USP, estrone and ethinyl estradiol, and salts, esters and prodrugs of any thereof.

6. The pharmaceutical gel composition of claim 4, further comprising at least one other pharmacologically active agent selected from the group consisting of progestogens and androgens.

7. The pharmaceutical gel composition of claim 6, wherein the progestogen is selected from the group consisting of progestogen, 17-hydroxy progestogen esters, 19-nor-17-hydroxy progestogen esters, norgestrel, norgestimate, desogestrel, demegestone, drospirenone, dydrogesterone, medrogestone, medroxy progesterone, medroxyprogesterone acetate, norethesterone, norethindrone, norethindrone acetate, levonorgestrel, 3-ketodesogestrel, gestodene and combinations thereof.

8. The pharmaceutical gel composition of claim 6, wherein the androgen is selected from the group consisting of testosterone, esters thereof, methyl-testosterone, prodrugs thereof and combinations thereof.

9. The pharmaceutical gel of claim 3, wherein the at least one pharmacologically active agent is a non-steroidal anti-inflammatory compound.

10. The pharmaceutical gel composition of claim 1, wherein the amount of the at least one pharmacologically active agent is from about 0.0025% to about 6% by weight of the composition.

11. The pharmaceutical gel composition of claim 10, wherein the amount of the at least one pharmacologically active agent is from about 0.0045% to about 5% by weight of the composition.

12. The pharmaceutical gel composition of claim 1, wherein the at least one hydrogen-bonding gelation polymer is selected from the group consisting of homopolymers, copolymers and interpolymers having pendant carboxylic acid groups, having pendant anhydrides of dicarboxylic acid groups or having both, and esters of any thereof.

13. The pharmaceutical gel composition of claim 1, wherein the at least one hydrogen-bonding gelation polymer is present in an amount sufficient to form a gel with a viscosity ranging from about 25 Pa·s to about 1000 Pa·s at 20° C.

14. The pharmaceutical gel composition of claim 1, wherein the at least one gelation promoter comprises an aqueous solution of the gelation promoter.

15. The pharmaceutical gel composition of claim 1, wherein the at least one gelation promoter is selected from the group consisting of polyhydric alcohols, polyglycols, and combinations thereof.

16. The pharmaceutical gel composition of claim 15, wherein the polyhydric alcohol is selected from the group consisting of 1,2-butanediol, 1,3-butanediol, 1,4-butanediol, butynediol, butenediol, diethylene glycol, ethylene glycol, glycerol, glycofurol, 1,2-hexanediol, 1,2,6-hexanetriol, 3-methyl-1,5-pentanediol, 2-methyl-1,3-propanediol, 1,9-nonanediol, 1,5-pentanediol, poly(vinyl alcohol), 1,3-propanediol, propylene glycol and combinations thereof.

17. The pharmaceutical gel composition of claim 15, wherein the polyglycol is selected from the group consisting of butyl glycol, butyl diglycol, butyl polyglycol, diethylene glycol dimethyl ether, diethylene glycol monomethyl ether, diethylene glycol diethyl ether, diethylene glycol monoethyl ether, dipropylene glycol, dipropylene glycol dimethyl ether, poloxamers, methyl diglycol, methyl triglycol, methyl tetraglycol, poly(ethylene glycol), poly(oxyethylene) alkyl ethers, poly(oxyethylene) alkyl esters, poly(propylene glycol), tetraethylene glycol dimethyl ether, triethylene glycol, triethyl glycol dimethyl ether, tripropylene glycol, glycol-silane copolymers and combinations thereof.

18. The pharmaceutical gel composition of claim 15, wherein the polyglycol is selected from the group consisting of polyoxyethylene, polyoxypropylene, a copolymer of polyoxyethylene and polyoxypropylene and combinations thereof.

19. The pharmaceutical gel composition of claim 1, wherein at least 25% of the pharmacologically active agent is dissolved in said composition at 15° C.

20. The pharmaceutical gel composition of claim 1, wherein at least 50% of the pharmacologically active agent is dissolved in said composition at 15° C.

21. The pharmaceutical gel composition of claim 1, wherein at least 75% of the pharmacologically active agent is dissolved in said composition at 15° C.

22. The pharmaceutical gel composition of claim 1, further comprising at least one pharmaceutically acceptable excipient.

23. A method of making a pharmaceutical gel composition for topical administration comprising the step of admixing at least one pharmacologically active agent in an amount of about 0.00001% to about 10% by weight of the composition, at least one hydrogen-bonding gelation polymer, and at least one gelation promoter or aqueous solution thereof in an amount effective to at least partially solubilize the pharmacologically active agent and to gel the polymer to form the pharmaceutical gel composition, wherein at least a portion of the pharmacologically active agent is dissolved in the composition at 15° C.

24. The method of claim 23, wherein the admixing step comprises (a) at least partially solubilizing the pharmacologically active agent in the gelation promoter or in an aqueous solution thereof to form an at least partially solubilized pharmacologically active agent preparation and (b) combining the at least partially solubilized pharmacologically active agent preparation with the hydrogen-bonding gelation polymer to form the pharmaceutical gel composition.

25. A pharmaceutical gel composition made according to the method of claim 23.

26. A method of topical administration of a pharmacologically active agent for a human or animal comprising the step of administering the pharmaceutical gel composition of claim 1 to an accessible body surface of the human or animal.
